(19) United States
(12) Patent Application Publication
(10) Pub. No.: US 2013/0095152 A1
(43) Pub. Date: Apr. 18, 2013

(54) METHODS AND COMPOSITIONS FOR THE TREATMENT OF SYMPTOMS OF COMPLEX REGIONAL PAIN SYNDROME

(71) Applicant: Curemark, LLC, Rye, NY (US)

(72) Inventor: Joan M. FALLON, Bronxville, NY (US)

(73) Assignee: CUREMARK, LLC, Rye, NY (US)

(21) Appl. No.: 13/705,763

(22) Filed: Dec. 5, 2012

Related U.S. Application Data
(63) Continuation of application No. 12/493,147, filed on Jun. 26, 2009, now abandoned.
(60) Provisional application No. 61/076,043, filed on Jun. 26, 2008.

(51) Int. Cl. A61K 38/54; C12Q 1/37

(52) U.S. Cl. CPC A61K 38/54; C12Q 1/37
USPC 424/400; 424/94.1; 424/94.63; 424/94.61; 424/94.6; 424/94.2; 424/94.21; 435/23; 435/201; 435/198; 435/219; 435/213

(57) ABSTRACT

A therapeutic composition for the treatment of the symptoms of complex regional pain syndrome and a method for preparing the therapeutic composition is disclosed. The therapeutic composition is a stable pharmaceutical composition comprising one or more digestive and/or pancreatic enzymes. The therapeutic composition may be manufactured by a variety of encapsulation technologies. Delivery of the therapeutic composition may be made orally, through injection, by adherence of a medicated patch or by other methods. Further, a method of using fecal chymotrypsin level as a biomarker for the presence of complex regional pain syndrome, or the likelihood of an individual to develop complex regional pain syndrome is disclosed.

### CROSS-REFERENCE TO RELATED APPLICATIONS

This application claims priority under 35 U.S.C. §119 to U.S. Provisional Application 61/076,043, filed Jun. 26, 2008, incorporated by reference in its entirety herein.

### TECHNICAL FIELD

This disclosure relates to a treatment for the symptoms of complex regional pain syndrome (CRPS), and more particularly, to the use of pharmaceutical compositions comprising one or more digestive enzymes, such as one or more pancreatic enzymes, in the treatment of the symptoms of complex regional pain syndrome. The disclosure also relates to a method of making pharmaceutical compositions comprising one or more digestive enzymes. The disclosure further relates to the use of an individual's fecal chymotrypsin level as a diagnostic marker for determining whether an individual has CRPS, as well as to predict whether an individual will be beneficially treated with the described pharmaceutical compositions.

### BACKGROUND

Dysautonomias can result in symptoms in which one or more areas of the body are innervated by the autonomic nervous system. While some dysautonomias are well known, other conditions have yet to be determined as a dysautonomia.

Symptoms of known dysautonomias include: palpitations, chest pain, tachycardia, excessive fatigue, severe fluctuations in blood pressure, excessive sweating, fainting, exercise intolerance, shortness of breath, visual disturbances including blurred vision, tunneling, and double vision, migraines, dizziness, insomnia, gastrointestinal problems including diarrhea, and constipation, bloody stools, fainting/near fainting, frequent urination, convulsions, and cognitive impairment. Other symptoms such as depression, dysthymia, obsessive compulsive tendencies, and difficulty with ambulation and other symptoms may also be a part of the dysautonomic picture.

Conditions such as familial dysautonomia (FD), also known also as Riley-Day syndrome, Parkinson's disease, Guillaine-Barre syndrome (GBS), Dopamine-b-Hydroxalase deficiency, baroreflex failure, Guillaine-Barre Syndrome, neuroblastoma and other tumors which affect the neuroendocrine system, Aromatic L-Amino Acid Decarboxylase deficiency, Tetrahydrobiopterin deficiency, Familial Paraganglioma syndrome, “Shy-Drager Syndrome,” also referred to as “Multiple System Atrophy” or MSA, Neurally Mediated Syncope, also known as Neurocardiogenic Syncope, fetal fatal insomnia (FFI), diabetic cardiovascular neuropathy, hereditary sensory and autonomic neuropathy type III (HSAN III), Menke's disease, monoamine oxidase deficiency states, and other disorders of dopamine metabolism, dysautonomic syndromes and disorders of the cardiovasular system, Chaga's disease, diabetic autonomic failure, and pure autonomic failure, are well known as conditions associated with or primarily due to a dysautonomia.

Complex regional pain syndrome (CRPS) is an uncommon, chronic condition that usually affects the arms or legs. Rarely, the disease can affect other parts of the body. Intense burning or aching pain may be experienced along with swelling, skin discoloration, altered temperature, abnormal sweating and hypersensitivity in the affected area. The nature of complex regional pain syndrome is puzzling, and the cause is not clearly understood.

Women are more likely to be affected by complex regional pain syndrome than men. Although complex regional pain syndrome is most common in people between the ages of 40 and 60, it can occur at any age.

Treatment for complex regional pain syndrome is most effective when started early in the course of the syndrome. Treatment options include:

Medications. Doctors use various medications to treat the symptoms of complex regional pain syndrome. Over-the-counter nonsteroidal anti-inflammatory drugs (NSAIDs), such as aspirin, ibuprofen (Advil, Motrin, others) and naproxen sodium (Aleve), may ease pain and inflammation. In some cases, doctors may recommend prescription medications. For example, antidepressants, such as amitriptyline and anticonvulsants such as gabapentin (Neurontin) are used to treat pain that originates from a damaged nerve (neuropathic pain). Cortico steroids, such as prednisone, may reduce inflammation.

Doctors may also suggest bone-loss medications, such as alendronate (Fosamax) and calcitonin (Miacalcin). Opioid medications may be another option. Taken in appropriate doses, they may provide acceptable control of pain. However, they may not be appropriate if you have a history of substance abuse or lung disease. Some pain medications, such as COX-2 inhibitors (Celebrex), may increase the risk of heart attack and stroke.

Applying heat and cold. Applying cold may relieve swelling and sweating. If the affected area is cool, applying heat may offer relief.

Capsaicin. This cream, made from the seeds of hot chili peppers, may relieve pain caused by nerve damage in early-stage complex regional pain syndrome. Doctors may recommend applying the cream to the affected area several times daily. Capsaicin cream can be very irritating if rubbed on nonaffected parts of your body. Follow the application instructions carefully. An individual should be able to tell within a week whether the treatment is effective and tolerable.

Physical therapy. Gentle, guided exercising of the affected limbs may improve range of motion and strength. The earlier the disease is diagnosed, the more effective exercises may be

Sympathetic nerve-blocking medication. Injection of an anesthetic to block pain fibers in your affected nerves may relieve pain in some people.

Transcutaneous electrical nerve stimulation (TENS). Chronic pain is sometimes eased by applying electrical impulses to nerve endings.

Biofeedback. In some cases, learning biofeedback techniques may help. In biofeedback, you learn to become more aware of your body so that you can relax your body and relieve pain.

Spinal cord stimulation. Your doctor inserts tiny electrodes along your spinal cord. A small electrical current delivered to the spinal cord sometimes results in pain relief.

The main symptom of complex regional pain syndrome is intense pain, often described as “burning.” Additional signs and symptoms include skin sensitivity and changes in skin temperature, color and texture. At times the skin may be sweaty; at other times it may be cold. Skin color can range from white and mottled to red or blue. Skin may become tender, thin or shiny in the affected area. Other symptoms include changes in hair and nail growth and joint stiffness, swelling and damage, muscle spasms, weakness and loss (atrophy), and decreased ability to move anaffected body part.

The illness may also spread from its source to elsewhere in the body in these patterns:

Continuity type. The symptoms may migrate from the initial site of the pain, for example, from the hand to the shoulder, trunk and face, affecting a quadrant of the body.

Mirror-image type. The symptoms may spread from one limb to the opposite limb.

Independent type. Sometimes, the symptoms may leap to a distant part of the body.

Complex regional pain syndrome typically has three stages, though not everyone progresses through these phases at the same pace:

Stage 1. Severe pain develops in one of the limbs. Swelling, sensitivity to touch or to cold, and skin changes, such as drying or thinning, begin to appear. This stage usually lasts one to three months.

Stage 2. Changes to the color and texture of the skin become increasingly obvious, and the swelling spreads. Stiffness in the muscles and joints may begin to be felt. This stage may last three to six months.

Stage 3. Severe damage is evident, such as limited movement in the affected limb, irreversible skin damage, muscle atrophy and contractures in nearby digits.

Complex regional pain syndrome occurs in two types with similar signs and symptoms, but different causes:

Type I. Previously known as reflex sympathetic dystrophy syndrome, this type occurs after an illness or injury that did not directly damage the nerves in the affected limb. About 90 percent of people with complex regional pain syndrome have type I.

Type II. Once referred to as causalgia, this type follows a distinct nerve injury. Many cases of complex regional pain syndrome occur after a forceful trauma to an arm or a leg, such as a gunshot wound or shrapnel blast. Other major and minor traumas, surgery, heart attacks, infections, fractures and even sprained ankles also can lead to complex regional pain syndrome. It is not well understood why these injuries sometimes trigger complex regional pain syndrome.

### SUMMARY

It has been determined by the present inventor that the gastrointestinal tract of dysautonomic individuals is impaired, and that the proper levels of pancreatic enzymes and/or their precursors including the zymogens and bicarbonate ions are not present in sufficient quantities to allow proper digestion. While that impairment is relevant to the digestion of carbohydrates, fats and proteins, it is most specific and most severe with respect to protein digestion. Accordingly, while not being bound by theory, the present inventor believes that many, if not all, dysautonomias have a GI component, and thus that dysautonomias may actually have their etiology in gastrointestinal dysfunction. For example, with Guillaine-Barre syndrome, it is postulated that a GI pathogen is a causative factor in the formation of the Guillaine Barre dysautonomia. Similarly, it has been found by the present inventor that populations of autistic children suffer from GI disturbances and other conditions which are dysautonomic in nature. In general, these findings represent a possible link between the etiology of autism and autonomic dysfunction. Thus, the inventor believes that other dysautonomic conditions also have GI primary etiologies.

The symptoms of dysautonomic conditions, however, may have various manifestations due to the genetic makeup of the individuals suffering from the conditions. Various gene sequences in the genetic code of the individual will result in manifestation of certain diseases or symptoms that are expressed uniquely in each individual. For example, if amino acid pool deficits due to improper protein digestion and gastrointestinal dysfunction are manifested differently in different individuals, a “disease state” may appear different depending upon the genetic makeup of the individual. Neurological expression may be all that is seen in some individuals, whereas other manifestations may demonstrate a hybrid of gastrointestinal dysfunction as well as neurological or other dysfunctions.

Accordingly, while not bound by theory, the present inventor believes that CRPS may have a dystautonomic component and that the etiology of CRPS may be related to gastrointestinal dysfunction.

Given the above, it is a goal of the present disclosure to provide therapeutic methods and pharmaceutical compositions for the treatment of the symptoms of complex regional pain syndrome. It is also a goal of the present disclosure to provide therapeutic methods and pharmaceutical compositions for the treatment of Pervasive Development Disorders such as Autism, ADD, and ADHD, and for Dysautonomias such as Familial Dysautonomia, Parkinson's, and Guillaine Barre Syndrome.

Another goal of the present disclosure is the provision of pharmaceutical compositions for the treatment of the above disorders, wherein the compositions comprise one or more digestive enzymes, e.g., one or more enzymes selected from amylases, proteases, cellulases, papaya, papain, bromelain, lipases, chymotrypsin, trypsin, and hydrolases. In some embodiments, the pharmaceutical compositions are lipid encapsulated.

Yet another goal of the present disclosure is to provide methods for making the described pharmaceutical compositions using methods such as: direct compression, microencapsulation, lipid encapsulation, wet granulation or other methods including the use of Prosolv® (silicified microcrystalline cellulose), and other known excipients and additives to accomplish microencapsulation, lipid encapsulation, direct compression, wet or dry granulation or other suitable technology.

A further goal of the present disclosure is to provide means to deliver the pharmaceutical compositions, which can include the use of rapid dissolution (rapid dissolve), time release, or other delivery methods including oral, injection, patch, or other method. Further, the delivery of the pharmaceutical compositions may be in the form of a tablet, capsule, sprinkles, sachet, or other oral delivery method.

An additional goal of the disclosure is to demonstrate the use of fecal chymotrypsin level as a biomarker for the presence of complex regional pain syndrome, or the likelihood of an individual to develop complex regional pain syndrome.

Accordingly, provided herein is a method for treating one or more symptoms associated with CRPS in a patient diagnosed with CRPS comprising administering to the patient a therapeutically effective amount of a pharmaceutical composition comprising one or more digestive enzymes. In some embodiments, the pharmaceutical composition may be lipid-encapsulated. In some embodiments, the one or more digestive enzymes comprise one or more enzymes selected from the group consisting of proteases, amylases, celluloses, sucrases, maltases, papaya, papain, bromelain, hydrolases, and lipases. In some embodiments, the one or more digestive enzymes comprise one or more pancreatic enzymes. In some embodiments, the pharmaceutical composition comprises one or more proteases, one or more lipases, and one or more amylases. In some embodiments, the one or more proteases comprise chymotrypin and trypsin.

The one or more digestive enzymes are, independently, derived from an animal source, a microbial source, or a plant source, or are synthetically prepared. In some embodiments, the animal source is a pig, e.g.: a pig pancreas.

In some embodiments, the pharmaceutical composition comprises at least one amylase, a mixture of proteases comprising chymotrypsin and trypsin, at least one lipase, and papain. In some embodiments, the pharmaceutical composition further comprises papaya. In some embodiments, the pharmaceutical composition comprises per dose: amylases from about 10,000 to about 60,000 U.S.P, proteases from about 10,000 to about 70,000 U.S.P, lipases from about 4,000 to about 30,000 U.S.P, chymotrypsin from about 2 to about 5 mg, trypsin from about 60 to about 100 mg, papain from about 3,000 to about 10,000 USP units, and papaya from about 30 to about 60 mg.

In some embodiments, the pharmaceutical composition comprises at least one protease and at least one lipase, wherein the ratio of total proteases to total lipases (in USP units) ranges from about 1:1 to about 20:1. In some embodiments, the ratio of proteases to lipases ranges from about 4:1 to about 10:1.

In some embodiments, the one or more symptoms of CRPS are selected from intense pain, a burning sensation, skin sensitivity, changes in skin temperature, color or texture, changes in hair and nail growth, joint stiffness, swelling and damage, muscle spasms, muscle weakness, muscle loss (atrophy), and decreased ability to move an affected body part.

In some embodiments, the pharmaceutical composition is a dosage formulation selected from the group consisting of: pills, tablets, capsules, microcapsules, mini-capsules, time released capsules, mini-tabs, sprinkles, and a combination thereof.

Also provided is a method of diagnosing a patient comprising: obtaining a fecal sample from the patient, determining a level of chymotrypsin present in the fecal sample, wherein the determination is performed at 30° C., and diagnosing the patient as having CRPS if the determined fecal chymotrypsin level is 8.4 U/gram or less and the patient exhibits at least one symptom associated with CRPS. In some embodiments, the fecal chymotrypsin level is between 8.4 and 4.2 U/gram. In some embodiments, the fecal chymotrypsin level is less than 4.2 U/gram. In some embodiments, the level of chymotrypsin present in the fecal sample is determined using an enzymatic photospectrometry method. In some embodiments, the method further comprises administering to the patient an effective amount of a pharmaceutical composition comprising one or more digestive enzymes if the patient is diagnosed as having CRPS. In some embodiments, the method further comprises determining if the administration of the pharmaceutical composition reduces one or more symptoms associated with CRPS.

Also provided is a method of identifying a patient likely to benefit from administration of a pharmaceutical composition comprising one or more digestive enzymes comprising: obtaining a fecal sample from the patient, determining a level of chymotrypsin present in the fecal sample, wherein the determination is performed at 30° C., and identifying the patient as likely to benefit from administration of the pharmaceutical composition if the determined fecal chymotrypsin level is 8.4 U/gram or less and the patient is diagnosed with CRPS. In some embodiments, the method further comprises determining if the patient exhibits one or more symptoms of CRPS. In some embodiments, the benefit comprises a reduction or amelioration of one or more symptoms associated with CRPS. In some embodiments, the method further comprises administering to the patient an effective amount of a pharmaceutical composition comprising one or more digestive enzymes.

Also provided is a pharmaceutical composition comprising one or more digestive enzymes, wherein the one or more digestive enzymes comprise at least one lipase and at least one protease, and wherein the ratio of total proteases to total lipases (in USP units) ranges from about 1:1 to about 20:1. In some embodiments, the ratio of total proteases to total lipases ranges from about 4:1 to about 10:1. In some embodiments, the pharmaceutical composition is lipid encapsulated.

Also provided is a pharmaceutical composition comprising at least one amylase, a mixture of proteases comprising chymotrypsin and trypsin, at least one lipase, and papain. In some embodiments, the pharmaceutical composition further comprises papaya. In some embodiments, the ratio of total proteases to total lipases ranges from about 1:1 to about 20:1.

The features and advantages described herein are not all-inclusive and, in particular, many additional features and advantages will be apparent to one of ordinary skill in the art in view of the specification, and claims. Moreover, it should be noted that the language used in the specification has been principally selected for readability and instructional purposes, and not to limit the scope of the inventive subject matter.

### DETAILED DESCRIPTION

The present disclosure provides pharmaceutical compositions and methods for treating symptoms associated with CRPS, Pervasive Development Disorders, and Dysautonomias. The pharmaceutical compositions described herein include one or more digestive enzymes, which are postulated by the present inventor to assist in proper digest protein and thus to ameliorate the gastrointestinal dysfunction that is associated with the described disorders.

In certain embodiments, the pharmaceutical compositions can include one or more digestive enzymes, wherein the one or more digestive enzymes comprise at least one lipase and at least one protease, and wherein the ratio of total proteases to total lipases (in USP units) ranges from about 1:1 to about 20:1. In some cases, the ratio of total proteases to total lipases ranges from about 4:1 to about 10:1.

In some cases, a pharmaceutical composition for use herein comprises at least one amylase, at least one protease, and at least one lipase. In certain embodiments, the pharmaceutical composition includes multiple proteases, including, without limitation, chymotrypsin and trypsin. In certain embodiments, the composition can further include one or more hydrolases, papain, bromelain, papaya, celluloses, pancreatin, sucrases, and maltases.

The one or more enzymes can be independently derived from animal, plant, microbial, or synthetic sources. In some embodiments, the one or more enzymes are derived from pig, e.g.: pig pancreas.

One exemplary formulation for the treatment of the symptoms of complex regional pain syndrome is as follows:
Amylase 10,000-60,000 U.S.P
Protease 10,000-70,000 U.S.P
Lipase 4,000-30,000 U.S.P
Chymotrypsin 2-5 mg
Trypsin 60-100 mg
Papain 3,000-10,000 USP units/mg
Papaya 30-60 mg

Additional formulations comprising one or more digestive enzymes may be advantageous including formulations in which the ratio of total proteases to total lipases (in USP units) is from about 1:1 to about 20:1. In some embodiments, the ratio of total proteases to total lipases is from about 4:1 to about 10:1. Such formulations are useful for treating symptoms of CRPS as well as dysautonomias (e.g., familial dysautonomia, Parkinson's, Guillaine-Barre Syndrome, Aromatic-L-amino acid decarboxylase deficiency, tetrahydrobiopterin deficiency, familial paranganglioma syndrome; multiple system atrophy, dysautonomic symptoms associated with tumors such as pheochromocytoma, chemodectoma, and neuroblastoma; neurally mediated syncope, and SIDS) and pervasive development disorders such as autism, ADHD, ADD, and Asperger's.

Patients below the age of 18 are typically given a dosage such that the formulation would deliver at least 5,000 USP units of protease and no more than 10,000 USP units of lipase per kilogram weight of patient, per day. Beneficially the formulation would deliver at least 5,000 USP units of protease and no more than 7,500 USP units of lipase per kilogram weight of patient per day. Patients above the age of 18 are typically given no less than 5,000 USP units of protease per kilogram weight of patient per day.

The dosage formulation may be administered by an oral preparation including, but not limited to, an encapsulated tablet, mini-tabs, microcapsule, mini-capsule, time released capsule, sprinkle or other methodology. In one embodiment, the oral preparation is encapsulated using lipid. Alternatively, the oral preparation may be encapsulated using enteric coating or organic polymers. A formulation may also be prepared using Prosolv® technology, direct compression, dry granulation, wet granulation, and/or a combination of these methods.

Fecal chymotrypsin level is a sensitive, specific measure of proteolytic activity, see e.g.: U.S. Pat. No. 6,660,831, incorporated by reference herein. Normal levels of chymotrypsin are considered be greater than 8.4 U/gram. Decreased values (less than 4.2 U/gram) suggest diminished pancreatic output (pancreatic insufficiency), hypoacidity of the stomach or cystic fibrosis. Elevated chymotrypsin values suggest rapid transit time, or less likely, a large output of chymotrypsin from the pancreas.

For the fecal chymotrypsin test, a stool sample is collected from each of the subjects. Each stool sample can be analyzed using an enzymatic photo spectrometry analysis to determine the level of fecal chymotrypsin in the stool; in some cases the assay is performed at 30° C., see e.g.: U.S. Pat. No. 6,660,831, incorporated by reference herein. Alternatively, other methods, such as the colorimetric method, use of substrates, use of assays, and/or any other suitable method may be used to measure the fecal chymotrypsin levels. The levels of fecal chymotrypsin in the samples of the individuals having complex regional pain syndrome are compared to the levels of fecal chymotrypsin in individuals not diagnosed with complex regional pain syndrome to determine if the individuals having complex regional pain syndrome would benefit from the administration of digestive enzymes.

The foregoing description of the embodiments of the disclosure has been presented for the purposes of illustration and description. It is not intended to be exhaustive or to limit the disclosure to the precise form disclosed. Many modifications and variations are possible in light of this disclosure. It is intended that the scope of the disclosure be limited not by this detailed description, but rather by the claims appended hereto.

What is claimed is:

1. A method for treating one or more symptoms associated with Complex Regional Pain Syndrome in a patient diagnosed with Complex Regional Pain Syndrome comprising administering to the patient a therapeutically effective amount of a pharmaceutical composition comprising one or more digestive enzymes.

2. The method of claim 1 wherein the one or more digestive enzymes comprise one or more enzymes selected from the group consisting of proteases, amylases, celluloses, sucrases, maltases, papaya, papain, and lipases.

3. The method of claim 1 wherein the one or more digestive enzymes comprise one or more pancreatic enzymes.

4. The method of claim 2 wherein the proteases comprise chymotrypin and trypsin.

5. The method of claim 1 wherein the one or more digestive enzymes are, independently, derived from an animal source, a microbial source, or a plant source, or are synthetically prepared.

6. The method of claim 5 wherein the animal source is a pig.

7. The method of claim 1 wherein the pharmaceutical composition comprises at least one amylase, a mixture of proteases comprising chymotrypsin and trypsin, at least one lipase, and papain.

8. The method of claim 7 wherein the pharmaceutical composition further comprises papaya.

9. The method of claim 1 wherein the pharmaceutical composition comprises: amylases from about 10,000 to about 60,000 U.S.P, proteases from about 10,000 to about 70,000 U.S.P, lipases from about 4,000 to about 30,000 U.S.P, chymotrypsin from about 2 to about 5 mg, trypsin from about 60 to about 100 mg, papain from about 3,000 to about 10,000 USP units, and papaya from about 30 to about 60 mg.

10. The method of claim 1 wherein the pharmaceutical composition comprises at least one protease and at least one lipase, and wherein the ratio of total proteases to total lipases (in USP units) ranges from about 1:1 to about 20:1.

11. The method of claim 10 wherein the ratio of proteases to lipases ranges from about 4:1 to about 10:1.

12. The method of claim 1 wherein the one or more symptoms of Complex Regional Pain Syndrome are selected from intense pain, a burning sensation, skin sensitivity, changes in skin temperature, color or texture, changes in hair and nail growth, joint stiffness, swelling and damage, muscle spasms, muscle weakness, muscle loss (atrophy), and decreased ability to move an affected body part.

13. The method of claim 1 wherein the pharmaceutical composition is a dosage formulation selected from the group consisting of: pills, tablets, capsules, microcapsules, mini-capsules, time released capsules, mini-tabs, sprinkles, and a combination thereof.
Complex Regional Pain Syndrome.

14. A method of diagnosing a patient comprising:
obtaining a fecal sample from the patient;
determining a level of chymotrypsin present in the fecal sample; and
diagnosing the patient as having Complex Regional Pain Syndrome if the determined fecal chymotrypsin level is 8.4 U/gram or less and the patient exhibits at least one symptom associated with.

15. The method of claim 14 wherein the fecal chymotrypsin level is between 8.4 and 4.2 U/gram.

16. The method of claim 14 wherein the fecal chymotrypsin level is less than 4.2 U/gram.

17. The method of claim 14 wherein the level of chymotrypsin present in the fecal sample is determined using an enzymatic photospectrometry method.

18. The method of claim 14 further comprising administering to the patient an effective amount of a pharmaceutical composition comprising one or more digestive enzymes if the patient is diagnosed as having Complex Regional Pain Syndrome.

19. The method of claim 18 further comprising determining if the administration of the pharmaceutical composition reduces one or more symptoms associated with Complex Regional Pain Syndrome.

20. The method of claim 19 further comprising comparing the post-administration measurement of one or more Complex Regional Pain Syndrome symptoms to a pre-administration measurement of the one or more Complex Regional Pain Syndrome symptoms.

21. A method of identifying a patient likely to benefit from administration of a pharmaceutical composition comprising one or more digestive enzymes comprising:
obtaining a fecal sample from the patient;
determining a level of chymotrypsin present in the fecal sample; and
identifying the patient as likely to benefit from administration of the pharmaceutical composition if the determined fecal chymotrypsin level is 8.4 U/gram or less and the patient is diagnosed with Complex Regional Pain Syndrome.

22. The method of claim 21 further comprising determining if the patient exhibits one or more symptoms of Complex Regional Pain Syndrome.

23. The method of claim 21 wherein the benefit comprises a reduction in one or more symptoms associated with Complex Regional Pain Syndrome.

24. The method of claim 21 wherein the level of chymotrypsin present in the fecal sample is determined using an enzymatic photospectrometry method.

25. The method of claim 21 further comprising administering to the patient an effective amount of a pharmaceutical composition comprising one or more digestive enzymes.

26. A pharmaceutical composition comprising one or more digestive enzymes, wherein the one or more digestive enzymes comprise at least one lipase and at least one protease, and wherein the ratio of total proteases to total lipases (in USP units) ranges from about 1:1 to about 20:1.

27. The pharmaceutical composition of claim 26 wherein the ratio of total proteases to total lipases ranges from about 4:1 to about 10:1.

28. A pharmaceutical composition comprising at least one amylase, a mixture of proteases comprising chymotrypsin and trypsin, at least one lipase, and papain.

29. The pharmaceutical composition of claim 28 wherein the pharmaceutical composition further comprises papaya.

30. The pharmaceutical composition of claim 28 wherein the ratio of total proteases to total lipases ranges from about 1:1 to about 20:1.

31. A pharmaceutical preparation for treating an individual exhibiting one or more symptoms of complex regional pain syndrome comprising a therapeutically effective amount of a digestive enzyme.

32. The pharmaceutical preparation of claim 31 wherein the digestive enzyme is selected from the group consisting of: amylase, lipase, protease, and a combination thereof.

33. The pharmaceutical preparation of claim 31 wherein the digestive enzyme is further selected from the group consisting of: chymotrypsin, trypsin, papaya, papain, and a combination thereof.

34. The pharmaceutical preparation of claim 31 wherein the enzyme is derived from a source selected from the group consisting of animal enzymes, plant enzymes, synthetic enzymes, and a combination thereof.

35. The pharmaceutical preparation of claim 31 wherein the preparation is manufactured using a technology selected from the group consisting of Prosolv® technology, enteric coating, lipid encapsulation, direct compression, dry granulation, wet granulation, and a combination thereof.

36. The pharmaceutical preparation of claim 31 wherein the preparation is administered orally via a dosage formulation selected from the group consisting of: pills, tablets, capsules, microcapsules, mini-capsules, time released capsules, mini-tabs, sprinkles, and a combination thereof.

37. The pharmaceutical preparation of claim 32 wherein the amount of amylase ranges from 10,000 to 60,000 USP units/mg.

38. The pharmaceutical preparation of claim 32 wherein the amount of protease ranges from 10,000 to 70,000 USP units/mg.

39. The pharmaceutical preparation of claim 32 wherein the amount of lipase ranges from 4,000 to 30,000 USP units/mg.

40. The pharmaceutical preparation of claim 33 wherein the amount of pancreatin ranges from 2,000 to 6,000 USP units/mg.

41. The pharmaceutical preparation of claim 33 wherein the amount of chymotrypsin ranges from 2 to 5 mg.

42. The pharmaceutical preparation of claim 33 wherein the amount of papain ranges from 3,000 to 10,000 USP units/mg.

43. The pharmaceutical preparation of claim 33 wherein the amount of papaya ranges from 30 to 60 mg.

44. The pharmaceutical preparation of claim 33 wherein the amount of trypsin ranges from 60 to 100 mg.

45. The pharmaceutical preparation of claim 31 wherein a symptom of the complex regional pain syndrome is ameliorated.

46. The pharmaceutical preparation of claim 31 wherein the symptom of the complex regional pain syndrome is selected from the group consisting of: pain, skin sensitivity, changes in skin temperature, color and texture, changes in hair and nail growth, joint stiffness, swelling and damage, muscle spasms, weakness and atrophy, decreased ability to move an affected body part, and a combination thereof.

47. A method of treating an individual having Complex Regional Pain Syndrome with a therapeutically effective amount of digestive enzymes comprising the steps of:
measuring a level of fecal chymotrypsin in a stool sample of the individual;
comparing the level of fecal chymotrypsin with a normal fecal chymotrypsin level; and
administering the digestive enzymes to the individual if the level of fecal chymotrypsin in the individual is less than the normal fecal chymotrypsin level.

48. The method of claim 47 further comprising the steps of:
administering the digestive enzymes to the individual in order to promote protein digestion; and
administering the digestive enzymes to the individual in order to ameliorate a symptom of the dysautonomic disorder.

49. The method of claim 47 wherein the stool sample is measured using a technique selected from the group consisting of: enzymatic photospectrometry, colorimetry, treatment with substrates, assays, and a combination thereof.
